(19) United States
(12) Patent Application Publication
(10) Pub. No.: US 2022/0031183 A1
(43) Pub. Date: Feb. 3, 2022

(54) BIOSIGNAL MEASUREMENT APPARATUS AND METHOD

(71) Applicant: Teemu MYLLYLÄ, Oulu (FI)

(72) Inventors: Teemu MYLLYLÄ, Oulu (FI); Aleksandra ZIENKIEWICZ, Oulu (FI)

(21) Appl. No.: 17/299,344

(22) PCT Filed: Dec. 3, 2019
(86) PCT No.: PCT/FI2019/050867

§ 371 Date: Jun. 3, 2021

(30) Foreign Application Priority Data
Dec. 4, 2018 (FI) 20186044

(51) Int. Cl. A61B 5/024; A61B 5/00; A61B 5/11

(52) U.S. Cl. CPC A61B 5/02438; A61B 5/6823; A61B 5/6804; A61B 5/02416; A61B 5/1107; A61B 5/6822

(57) ABSTRACT

A biosignal measurement apparatus comprises a data processing unit, and a first sensor, which is attachable to a surface of a body section at or adjacent to ribs and/or a neck and configured to convert movement pulses of the body section caused by muscular work of a heart at systolic and diastolic phases into pulses of an electric signal. The data processing unit receives the electric signal from the first sensor, and forms and outputs data about blood pressure on the basis of a characteristic feature of at least one of the movement pulses associated with the systolic phase and/or a characteristic feature of at least one of the movement pulses associated with the diastolic phase.

### FIELD

The invention relates to a biosignal measurement apparatus and a biosignal measurement method.

### BACKGROUND

Blood pressure and a pulse rate may be measured using a manual or digital sphygmomanometer which has a cuff placed around an arm and a pressure meter. A manual measurement of the blood pressure may be based on an auscultation with a stethoscope. An automatic digital measurement of the blood pressure may determine the systolic and diastolic pressures on the basis of oscillometric detection.

The blood pressure has also been measured using a photoplethysmography meter that optically detects light absorption in a tissue, the light absorption varying with the cardiac cycle. Also the photoplethysmography meter, which is often attached to a fingertip like a clothespin, requires a considerable pressure against the skin for a good contact and a reliable measurement. These same devices may also measure the heart rate.

In general, to detect the pressure variation in the artery and derive also the heart rate therefrom is still challenging. Additionally, the pressure of the cuff of the sphygmomanometer and the photoplethysmography meter for detecting the pressure variation in the artery cause stress and pain to a measured person, which is unpleasant and can even result in error. The measurement time is also limited because of the specific use and the uncomfortable pressure. Hence, there is a need to improve the biosignal measurements.

### BRIEF DESCRIPTION

The present invention seeks to provide an improvement in the biosignal measurements.

The invention is defined by the independent claims. Embodiments are defined in the dependent claims.

### LIST OF DRAWINGS

Example embodiments of the present invention are described below, by way of example only, with reference to the accompanying drawings, in which

FIG. 1 illustrates an example of a biosignal measurement apparatus on a surface of a body section;

FIG. 2 illustrates an example of a biosignal measurement apparatus;

FIG. 3 illustrates an example of an electric signal with movement pulses;

FIG. 4 illustrates an example of an electric signal with movement pulses and an electrocardiogram on the same time scale;

FIG. 5 illustrates an example of an electric signal with movement pulses and a photoplethysmogram;

FIG. 6 illustrates an example of a data processing unit of the biosignal measurement apparatus and its potential connection to an external device; and

FIG. 7 illustrates of an example of a flow chart of a biosignal measuring method.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may contain also features/structures that have not been specifically mentioned. All combinations of the embodiments are considered possible if their combination does not lead to structural or logical contradiction.

It should be noted that while Figures illustrate various embodiments, they are simplified diagrams that only show some structures and/or functional entities. The connections shown in the Figures may refer to logical or physical connections. It is apparent to a person skilled in the art that the described biosignal measurement apparatus may also comprise other functions and structures than those described in Figures and text. It should be appreciated that details of some functions, structures, and the signalling used for measurement and/or controlling are irrelevant to the actual invention. Therefore, they need not be discussed in more detail here.

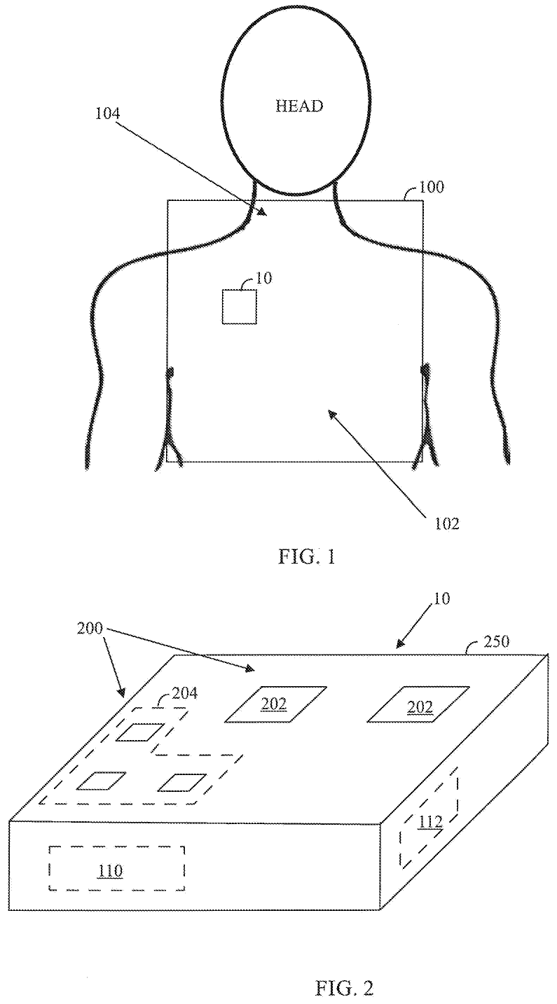

FIG. 1 illustrates an example of a biosignal measurement apparatus 10 located on a surface of a body section 100. The biosignal apparatus 10 may be attached to or adjacent to ribs 102. Additionally or alternatively, the biosignal apparatus may be attached to a neck 104. The body section 100 may comprise a chest and a frontal section of the neck 104. The frontal section of the neck 104 refers to an area of the neck that can be seen when standing in front of a person and facing the person. The body section 100 may comprise a back at or adjacent to the ribs 102 and a back section of the neck 104. In an embodiment, the biosignal measurement apparatus 10 is located on the chest 102. In an embodiment, the biosignal measurement apparatus 10 is located on the frontal section of the neck 104. The location of the biosignal measurement apparatus may be freely selected within the borders of body section 100.

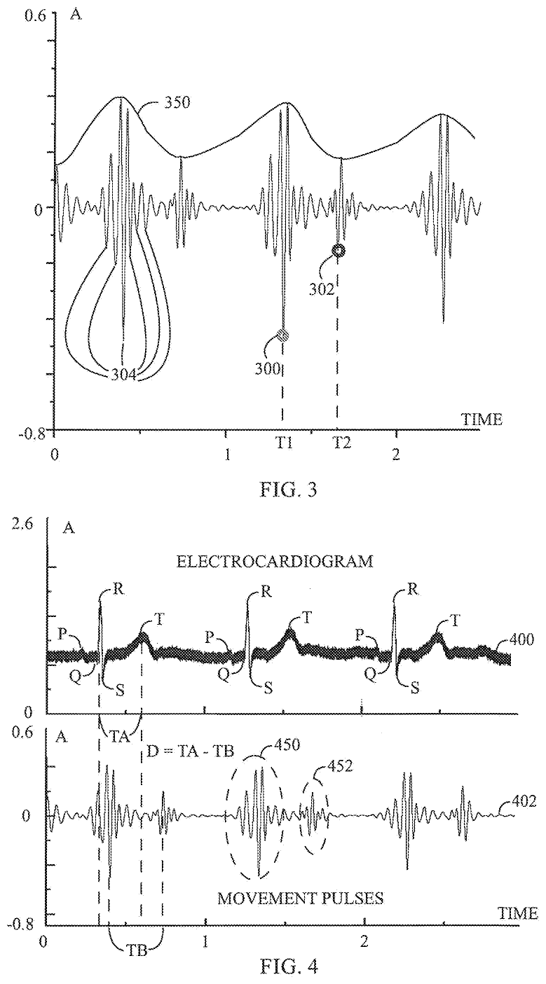

FIG. 2 illustrates an example of the biosignal measurement apparatus 10 that comprises a data processing unit 110 and a first sensor 112, which both may be located on or within a case 250 of the biosignal measurement apparatus 10. The apparatus 10 is attachable to the surface of the body section 100 and the first sensor 112 converts movements of the body section 100 into an electric signal. The movements of the body section 100 are caused by systolic and diastolic phases of the cardiac cycle of the heart and they appear as physical movement pulses. That is, the movement pulses of the body section 100, which occur in a repeated manner following the cardiac cycle of the heart, are directly caused by a muscular work of the heart which is independent from a movement caused by changes of blood pressure in veins such as arteries.

The systolic phase of the heart refers to a contraction of the heart, which also results in a maximum arterial blood pressure. The diastolic phase of the heart, in turn, refers to a muscle expansion of the heart, which occurs between heart beats and which results in a lower blood pressure than that of the systolic blood pressure. The muscle expansion may also be considered as muscle elongation or muscle relaxation. The contraction and the expansion of the heart muscles make the chest 102 and the frontal section of the neck 104 to move according to the movements having corresponding pulses.

The data processing unit 110 receives the electric signal that carries information about the movement pulses, from the first sensor 112. The data processing unit 110 then forms and outputs data about blood pressure on the basis of a characteristic feature of at least one of the movement pulses associated with the systolic phase and/or a characteristic feature of at least one of the movement pulses associated with the diastolic phase. A plurality of formed data about the blood pressure may be combined for forming statistical data about the blood pressure. The combined data may represent at least one average blood pressure, for example. The combined data may represent an average systolic blood pressure, for example. The combined data may represent an average diastolic blood pressure, for example.

In an embodiment, the movement pulses of the systolic phase and the diastolic phase may be from a common heartbeat. In an embodiment, the movement pulses of the systolic phase and the diastolic phase may be from different heartbeats which have a deterministic or known interdependence therebetween. For example, two heartbeats that have a heartbeat therebetween may be similar enough for the measurement. Correspondingly, two heartbeats that have many heartbeats therebetween may be similar enough for the measurement, particularly if the conditions for or a condition of the person that is measured do not change.

In an embodiment, the first sensor 112 may comprise an acceleration sensor that is known, per se, to a person skilled in the art. The acceleration sensor may be mechanical, piezoelectric (resistive or capacitive) or optical, for example. The movement pulses can be measured as acceleration and/or variation of the acceleration.

In an embodiment, the first sensor 112 may comprise a microphone. The microphone, which is known, per se, to a person skilled in the art, may be based on resistance, capacitance, inductance or piezoelectric phenomenon. The first sensor 112 may also be called a vibration sensor or a phonometer. In this manner, the blood pressure may be measured by concentrating on the effects caused directly by a muscular work of the heart without actually measuring any artery pressure.

In an embodiment, the data processing unit 110 may determine the blood pressure on the basis of a first movement pulse 450 that corresponds to a systolic phase of the heart and a second movement pulse 452 that corresponds to a diastolic phase of the heart (see FIG. 4). The movement pulse 450 of the systolic phase may be stronger than the movement pulse 452 of the diastolic phase, which may be used to recognize the movement pulses 450, 452 from each other. Alternatively, an electrocardiogram may be utilized for that.

In an embodiment, the data processing unit 110 forms the data about the blood pressure on the basis of a characteristic feature of a movement pulse of the systolic phase. In an embodiment, the data processing unit 110 forms the data about the blood pressure on the basis of a characteristic feature of a movement pulse of the diastolic phase. Each of the differences may be measured within one cardiac cycle or more than one cardiac cycles having known interrelation therebetween.

In an embodiment an example of which is illustrated in FIG. 3, the characteristic feature may be an absolute maximum amplitude 300 of a movement pulse of the systolic phase. In an embodiment the characteristic feature may be an absolute maximum amplitude 302 of a movement pulse of the diastolic phase. The vertical axis is amplitude A in arbitrary units and the horizontal axis is time in seconds. Here the term “absolute” refers to non-negative real values.

In an embodiment, the characteristic feature may be an area of a movement pulse of the systolic phase. In an embodiment, the characteristic feature may be an area of a movement pulse of the diastolic phase. The area of the movement pulse of the systolic phase may be computed by integrating an absolute function of the movement pulse of the systolic phase over a duration of the movement pulse of the systolic phase. Because the movement pulses may comprise a plurality of sub-pulses 304 in a bipolar manner (there are a plurality of sub-pulses in FIG. 3), the area of the movement pulses may be determined using only one sub-pulse or more than one sub-pulse.

In an embodiment, the characteristic feature may be a shape of a movement pulses of the systolic phase. In an embodiment, the characteristic feature may be a shape of a movement pulse of the diastolic phase. The shape may be determined by height of peaks of the sub-pulses. The shape may be determined by relation of and/or difference between the heights of peaks of the sub-pulses. The shape may be determined by the area occupied by the one or more sub-pulses.

In an embodiment, the data processing unit 110 forms the data about the blood pressure on the basis of a difference between a characteristic feature of a movement pulse of the systolic phase and a characteristic feature of a movement pulse of the diastolic phase. Each of the differences may be measured within one cardiac cycle or more than one cardiac cycles having known interrelation therebetween.

In an embodiment an example of which is illustrated in FIG. 3, the characteristic feature may be a difference between an absolute maximum amplitude 300 of a movement pulse of the systolic phase and an absolute maximum amplitude 302 of a movement pulse of the diastolic phase.

In an embodiment, the characteristic feature may be a difference between an area of a movement pulse of the systolic phase and an area of a movement pulse of the diastolic phase.

In an embodiment, the characteristic feature may be a difference between a shape of a movement pulses of the systolic phase and a shape of a movement pulse of the diastolic phase.

In an embodiment, the characteristic feature may be a difference between a moment T1 of occurrence of a movement pulse of the systolic phase and a moment T2 of occurrence of a movement pulse of the diastolic phase. In an embodiment, the moment T1 of the movement pulse of the systolic phase may be based on a moment of a maximum amplitude. In an embodiment, the moment T1 of the movement pulse of the systolic phase may be based on a moment of a middle point of a duration of the movement pulse of the systolic phase. In an embodiment, the moment T1 of the movement pulse of the systolic phase may be based on a moment of a beginning point or an end point of the movement pulse of the systolic phase. In an embodiment, the moment T1 of the movement pulse of the diastolic phase may be based on a moment of a maximum amplitude. In an embodiment, the moment T1 of the movement pulse of the diastolic phase may be based on a moment of a middle point of a duration of the movement pulse of the diastolic phase. In an embodiment, the moment T1 of the movement pulse of the diastolic phase may be based on a moment of a beginning point or an end point of the movement pulse of the diastolic phase. The beginning point may be where an absolute value of the movement pulse is and/or becomes larger than a first threshold. The end point may be where an absolute value of the movement pulse is and/or becomes smaller than a second threshold. The first and second thresholds may be the same or different. The first and/or second threshold(s) may be adaptive.

In an embodiment, the apparatus may also comprise a second sensor unit 200, which provides cardiac data on the basis of electromagnetism.

The cardiac data, in general, may include an electrocardiogram, information about pulses per time unit such as heart rate, and/or information about intervals between heart beats such as beat-to-beat intervals. The cardiac data may be described using a frequency domain variable, i.e. the heart rate, the cardiac data may also be based on a time-domain approach, i.e., the heart beat intervals.

In an embodiment, the second sensor unit 200 comprises at least one electrocardiogram sensor 202 that is known, per se, to a person skilled in the art. The electrocardiogram sensor 202 may have one or more sensor elements (FIG. 2 shows two elements merely as an example).

In an embodiment, the second sensor unit 200 comprises at least one photoplethysmogram sensor 204 that is known, per se, to a person skilled in the art. The photoplethysmogram sensor 204 may have one or more sensor elements (FIG. 2 shows three sensor elements merely as an example). The photoplethysmogram sensor 204 is in principle a pulse oximeter which has an optical radiation source, which illuminates towards the skin, and an optical radiation detector, which detects optical radiation scattered from the skin illuminated by the optical radiation source. The photoplethysmogram sensor 204 then measures optical power received by the detector. The optical power varies in a cyclic manner with the cardiac cycle.

Both the first sensor 110 and the second sensor unit 200 are attachable to the surface of the body section 100. FIG. 4 illustrates example of the electrocardiogram as the cardiac data 400 from the second sensor unit 200, and the movement pulses 402 from the first sensor 110. The cardiac data 400 may come from the electrocardiogram sensor 202 or from the photoplethysmogram sensor 204 (in FIG. 4, cardiac data 400 is from electrocardiogram sensor as an example). The vertical axes are in arbitrary scales, and the horizontal axes are time in seconds in FIG. 4. In an embodiment, the data processing unit 110 may receive the cardiac data 400 and form the data about the blood pressure on the basis of a moment TA of a heartbeat of the cardiac data 400 and a moment TB of the movement pulses 402. The moment TB may be related to a systolic phase or to a diastolic phase. In an embodiment, the moment TA may be related to R-peak of the QRS-complex of the electrocardiogram. In an embodiment, the moment TA may be related to T-peak of the QRS-complex of the electrocardiogram. In an embodiment, the moment TA of the cardiac data 400 may correspond to the moment TB of the movement pulses 402, and vice versa, such that they relate to the same muscular work phase of the heart. The cardiac data 400 shows also Q- and S-peaks, and additionally P- and T-peaks which may be used in determination of the blood pressure.

The moments TA and TB may be selected freely in the movement pulses and the heartbeat of the cardiac data as long as the selection is cardiac cyclically deterministic which is clear, per se, to a person skilled in the art. The processing unit 110 may form the data about the blood pressure on the basis of a delay D between the moment TA of the cardiac data 400 and the moment TB of the movement pulses 402 (D=TA−TB or D=TB−TA), for example.

In an embodiment, the data processing unit 110, the first sensor 112 and the potential second sensor unit 200 are integrated together and covered with a common case 250. The covering of the case 250 may include all parts except the sensor elements such as electrodes of the electrocardiogram sensor 202 which may have to be in contact with the skin. In an embodiment, the optical radiation source and the optical radiation detector of the at least one photoplethysmogram sensor 204 may be covered with a transparent material of the case. In an embodiment, however, the optical radiation source and the optical radiation detector of the at least one photoplethysmogram sensor 204 may be located at a hole of the case 250 which may be opaque.

In an embodiment an example which is illustrated in FIG. 2, the second sensor unit 200 comprises both the electrocardiogram sensor 202 and the photoplethysmogram sensor 204. The data processing unit 110 may receive a first piece of the cardiac data from the electrocardiogram sensor 202 and a second piece of the cardiac data from the photoplethysmogram sensor 204. The data processing unit 110 may then form the data about the blood pressure on the basis of a moment of the first piece of the cardiac data of a heartbeat and a moment of the second piece of the cardiac data of a heartbeat.

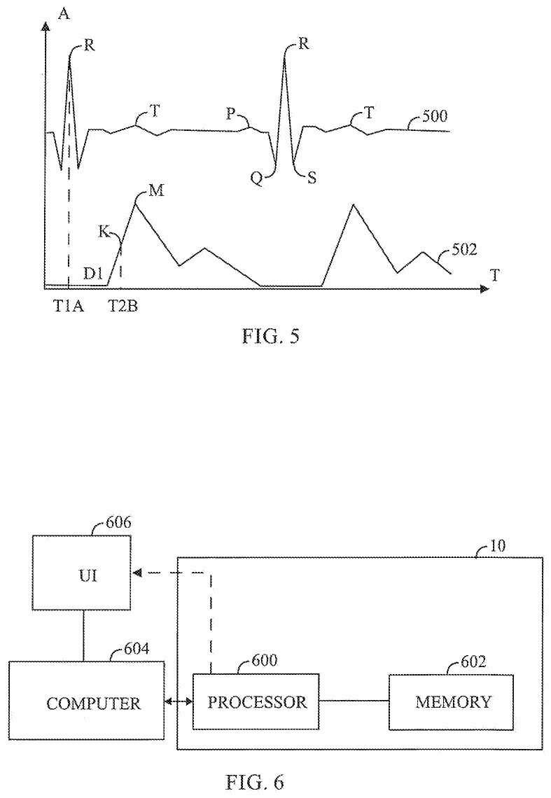

In an embodiment an example of which is illustrated in FIG. 5, the processing unit 110 may form the data about the blood pressure on the basis of a delay D1 between a moment T1A of the heartbeat of the first piece of the cardiac data 500 from the electrocardiogram sensor 202 and a corresponding moment T2B of the heartbeat of the second piece of the cardiac data 502 from the photoplethysmogram sensor 204. The vertical axis is amplitude A in an arbitrary scale and the horizontal axis is time T in an arbitrary scale. This delay measurement is similar to the measurement between the cardiac data and the movement pulses illustrated in FIG. 4.

The moments T1A, T2B may be related to R-peak of the QRS-complex of the electrocardiogram. The first piece of the cardiac data 500 shows also Q- and S-peaks, and additionally P- and T-peaks.

In an embodiment, the moment T2B corresponding to the R-peak in the second piece of the cardiac data 502 from the photoplethysmogram sensor 204 may be defined as a point at which there is a local maximum M. In an embodiment, the moment T2B corresponding to the R-peak in the second piece of the cardiac data 502 from the photoplethysmogram sensor 204 may be defined as a point K at which the slope has a highest value, for example. The highest value of the slope may be found by determining a function that has the same values as the second piece of the cardiac data 502, and then searching for a point where a derivative of said function is at its local maximum, for example. A person skilled in the art may find other corresponding points between the first piece of the cardiac data 500 and the second piece of the cardiac data 502 on the basis of which the blood pressure can be determined without actually measuring any artery pressure.

In an embodiment an example of which is illustrated in FIG. 3, the data processing unit 110 may form an envelope curve 350 of the movement pulses of the body section 100 and determine a characteristic feature of the at least one of the movement pulses associated with the systolic phase and a characteristic feature of at least one of the movement pulses associated with the diastolic phase on the basis of the envelope curve 350. The characteristic feature may relate to timing, a pulse height like amplitude or a pulse area as already explained earlier in association with FIG. 3.

FIG. 6 illustrates an example of the processing unit 110. In an embodiment, the processing unit 110 may comprise one or more processors 600, and one or more memories 602 including computer program code. The one or more memories 602 and the computer program code may, with the one or more processors 600, cause the biosignal measurement apparatus 10 at least to process the electric signal of the movement pulses. Additionally, the one or more memories 602 and the computer program code may, with the one or more processors 600, cause the biosignal measurement apparatus to process the cardiac data alone or together with the electric signal of the movement pulses. The one or more memories 602 may store the processed electrical signal, cardiac data and/or the formed data of the blood pressure. The biosignal measurement apparatus 10 may be in wireless or wired connection with a computer 604 that may gather the information from the biosignal measurement apparatus 10. The wireless connection may require a transmitter or a transceiver in the biosignal measurement apparatus 10. The wired connection requires a connector that is suitable with a counterpart connector of the computer 604. A user interface 606 may be in connection with the computer 604 such that the computer 604 can be used. Additionally, it may be possible to connect the biosignal measurement apparatus 10 directly to the user interface without any computer 604.

In an embodiment, the biosignal measurement apparatus 10 may have a user interface 606 of its own.

The user interface 606 may have a screen for showing the electrical signal, the cardiac data and/or the formed data of the blood pressure, and keys or a keyboard for inputting alphanumerical information and/or selecting operations from icons on the screen. Alternatively or additionally, the interface 606 may have a touch screen for showing the information and the input and/or the selection.

The apparatus 10 may be taped or attached using a belt on the skin of the body section 100 such that the at least one sensor element of the second sensor unit 200 is in contact with the skin of the body section 100. The apparatus 10 may also be integrated into a cloth or necklace or the like which allows the at least one sensor element of the second sensor unit 200 to be in contact with the skin of the body section 100.

The biosignal measurement apparatus 10 may reside in or attached to a user wearable structure. The user wearable structure may be a belt, a string or a band worn around the chest 102 or the neck 104. The user wearable structure may be a garment such as a shirt, a scarf, a top, a bra, or the like. The biosignal measurement apparatus may be in the case 250 the measures of which are H×B×W, where H may be in a range 1 cm to 3 cm, B may be in a range 2 cm to 4 cm and W may be in a range 0.5 cm to 2 cm, for example.

The biosignal measurement apparatus 10 may be utilized in hospitals, health care facilities, operating rooms or at home. The biosignal measurement apparatus 10 may used continuously for a long time. The person whose blood pressure and potentially other biosignal are measured with the biosignal apparatus 10 may be lying on his/her back or may be in inclined position such that a longitudinal axis of the upper section of the body such as the torso has a horizontal component while a line going through shoulders has also a horizontal component. However, the position of the measured person is not important. The measured person may stay still i.e. may not be walking, running, jumping or rotating/turning.

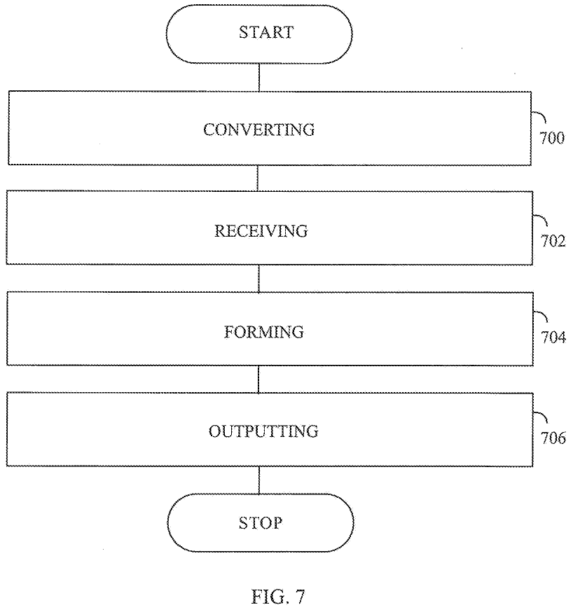

FIG. 7 is a flow chart of the biosignal measurement method. In step 700, movement pulses of a body section 100 caused by systolic and diastolic phases of a heart are converted to an electric signal by a first sensor 112, the first sensor 112 being attachable to a surface of the body section 100 comprising a chest 102 and a frontal section of a neck 104. In step 702, the electric signal from the first sensor 112 is received by the data processing unit 110. In step 704, data about blood pressure is formed on the basis of a characteristic feature of at least one of the movement pulses associated with a systolic phase and a characteristic feature of at least one of the movement pulses associated with a diastolic phase by the data processing unit 110. In step 706, the data about the blood pressure is output from the data processing unit 110.

The method shown in FIG. 7 may be implemented as a logic circuit solution or computer program. The computer program may be placed on a computer program distribution means for the distribution thereof. The computer program distribution means is readable by a data processing device, and it encodes the computer program commands and carries out the measurements. The computer program may also have instructions for controlling the biosignal measurement apparatus.

The computer program may be distributed using a distribution medium which may be any medium readable by the controller. The medium may be a program storage medium, a memory, a software distribution package, or a compressed software package. In some cases, the distribution may be performed using at least one of the following: a near field communication signal, a short distance signal, and a telecommunications signal.

It will be obvious to a person skilled in the art that, as technology advances, the inventive concept can be implemented in various ways. The invention and its embodiments are not limited to the example embodiments described above but may vary within the scope of the claims.

1. A biosignal measurement apparatus, wherein the biosignal measurement apparatus comprises a data processing unit, and a first sensor, which is attachable to a surface of a body section at or adjacent to ribs and/or a neck and configured to convert movement pulses of the body section directly caused by muscular work of a heart at systolic and diastolic phases into pulses of an electric signal, the movement pulses of the body section directly caused by the muscular work of the heart being independent from a movement caused by changes of blood pressure in veins; and
the data processing unit is configured to receive the electric signal from the first sensor, and form and output data about blood pressure on the basis of a characteristic feature of at least one of the movement pulses associated with the systolic phase and/or a characteristic feature of at least one of the movement pulses associated with the diastolic phase.

2. The biosignal measurement apparatus of claim 1, wherein the data processing unit is configured to form the data about the blood pressure on the basis of a characteristic feature of a movement pulse of the systolic phase, a characteristic feature of a movement pulse of the diastolic phase, or a difference therebetween, the difference being measured within one cardiac cycle or more than one cardiac cycles having known interrelation therebetween.

3. The biosignal measurement apparatus of claim 2, wherein the characteristic feature is a difference between at least one of the following:
an absolute maximum amplitude of a movement pulse of the systolic phase and an absolute maximum amplitude of a movement pulse of the diastolic phase, p1 an area of a movement pulse of the systolic phase and an area of a movement pulse of the diastolic phase,
a shape of a movement pulse of the systolic phase and a shape of a movement pulse of the diastolic phase, and
a moment of a movement pulse of the systolic phase and a moment of a movement pulse of the diastolic phase.

4. The biosignal measurement apparatus of claim 1, wherein the biosignal measurement apparatus comprises a second sensor unit configured to a measure cardiac data about the heart on the basis of electromagnetism, both the first sensor and the second sensor unit being attachable to the surface of the body section; and
the data processing unit is configured to receive the cardiac data and form the data about the blood pressure on the basis of a moment of the movement pulses and a moment of the cardiac data.

5. The biosignal measurement apparatus of claim 4, wherein the data processing unit, the first sensor and the second sensor unit are integrated together and covered with a common case.

6. The biosignal measurement apparatus of claim 4, wherein the second sensor unit comprises at least one of the following: an electrocardiogram sensor and a photoplethysmogram sensor.

7. The biosignal measurement apparatus of claim 5, wherein the second sensor unit comprises both the electrocardiogram sensor and the photoplethysmogram sensor; the data processing unit is configured to receive a first piece of the cardiac data from the electrocardiogram sensor and a second piece of the cardiac data from the photoplethysmogram sensor; and
the data processing unit is configured to form the data about the blood pressure on the basis of a moment of the first piece of the cardiac data and a moment of the second piece of the cardiac data.

8. The biosignal measurement apparatus of claim 6, wherein processing unit is configured to form the data about the blood pressure on the basis of a delay between the moment of the cardiac data and the moment of the movement pulses, the moment of the second piece and the moment of the movement pulses, or a delay between the moment of the first piece of the cardiac data and the moment of the second piece.

9. The biosignal measurement apparatus of claim 1, wherein the data processing unit. configured to form an envelope curve of the movement pulses of the body section and determine the characteristic feature on the basis of the envelope curve.

10. A biosignal measurement method, the method comprising
converting, by a first sensor, movement of a body section directly caused by systolic and diastolic phases of a heart to an electric signal, the first sensor being attachable to a surface of the body section at or adjacent to ribs and/or a neck, the movement pulses of the body section directly caused by the muscular work of the heart being independent from a movement caused by changes of blood pressure in veins; and
receiving, by the data processing unit, the electric signal from the first sensor;
forming, by the data processing unit, data about blood pressure on the basis of a characteristic feature of at least one of the movement pulses associated with a systolic phase and/or a characteristic feature of at least one of the movement pulses associated with a diastolic phase; and
outputting, from the data processing unit, the data about the blood pressure.
